(19) United States
(12) Patent Application Publication
(10) Pub. No.: US 2022/0387479 A1
(43) Pub. Date: Dec. 8, 2022

(54) NOVEL POVIDONE-IODINE PHARMACEUTICAL PREPARATION AND USES THEREOF

(71) Applicant: Purdue Pharma L.P., Stamford, CT (US)

(72) Inventors: David IGO, Raleigh, NC (US); Rosa HONG, Closter, NJ (US); Zachary Thomas ROHR, Chapel Hill, NC (US)

(73) Assignee: Purdue Pharma L.P., Stamford, CT (US)

(21) Appl. No.: 17/824,516

(22) Filed: May 25, 2022

Related U.S. Application Data
(60) Provisional application No. 63/192,830, filed on May 25, 2021.

(51) Int. Cl. A61K 31/79; A61K 47/10; A61K 9/00; A61P 31/04; A61K 47/26; A61K 33/18; A61K 47/38; A61P 31/12

(52) U.S. Cl. CPC A61K 31/79; A61K 47/10; A61K 9/0014; A61P 31/04; A61K 47/26; A61K 33/18; A61K 47/38; A61P 31/12

(57) ABSTRACT

This disclosure provides novel aqueous povidone-iodine (PVP-I) pharmaceutical preparations, which demonstrate strong antimicrobial activity and also strong virucidal activities. The disclosure also provides methods for reducing virus transmission or decreasing risk, incidence or severity of diseases (e.g., COVID-19) or conditions caused by virus infections by topically applying the pharmaceutical preparation of the disclosure to an animal (e.g., a human subject). The disclosure further provides methods of reducing an infection risk posed by microorganisms or viruses in a clinical setting.

### FIELD OF THE INVENTION

This disclosure relates to novel povidone-iodine (PVP-I) preparations, which demonstrate strong antimicrobial activity as well as strong virucidal activities, and method of uses thereof.

### BACKGROUND OF THE INVENTION

Polyvinylpyrrolidone—iodine (povidone—iodine or “PVP-I”) is an antiseptic agent with broad-spectrum anti-infective activity against a variety of pathogenic microorganisms. The efficacy and tolerability profile of PVP-I compared with that of other agents, such as chlorhexidine gluconate, polyhexanide, and octenidine, has been well established (Barreto R. et al., Int. J. Antimicrob Agents, 2020; 56:106084; Eggers M., Infect Dis. Ther. 2019; 8:581-93; Lachapelle J-M et al., Future Medicine, 2013; 10:579-92; & Kampf G. et al., J. Hosp Infect., 2020; 104:246-51). Regardless of clinical use spanning decades, there has been no or low documented resistance or cross-resistance (Barreto R. et al.; Eggers M.; Lachapelle J-M et al; & Williamson DA et al., BMC Infect. Dis., 2015; 15:375). The U.S. Food and Drug Administration confirmed the position in its final rules on nonprescription antiseptic preparations intended for professional use in healthcare settings by not requiring resistance testing for certain active ingredients, including PVP-I (Federal Register, 2017; 82:60474-60503). The salient feature of PVP-I adds utility in the era of antimicrobial resistance. Various PVP-I formulations have been developed, including oral rinses, gargles, topical solutions, and surgical scrubs, and are available for topical or oral use in healthcare, dental, and household settings.

Emergence of the novel severe acute respiratory syndrome coronavirus 2 (SARS-CoV-2) in 2019 and its spread globally over the following year have proven to be an unprecedented public health crisis (see, e.g., data in Johns Hopkins Univ., Coronavirus Res. Center). SARS-Cov-2 viruses may cause respiratory illness ranging from mild disease to severe disease and death in animals (including humans). The modes of transmission for SARS-CoV-2 viruses, including contact, droplet, airborne, formite, fecal-oral, bloodborne, mother-to-child, human-to-human and animal-to-human transmission, and like. Transmission sARS-CoV-2 can occur through direct, indirect, or close contact with infected people through infected secretions such as saliva and respiratory secretions or their respiratory droplets, which are expelled when an infected person coughs, sneezes, talks or sings (see, e. g., Transmission of SARS-CoV-2:implications for infection prevention precaution, WHO Scientific Brief dated 9 Jul. 2020).

Thus, there is a need for the development of novel PVP-I formulations which not only demonstrate broad-spectrum anti-infective activity against pathogenic microorganisms but also are highly effective in mitigating transmission of SARS-COV2 viruses and/or reducing infection risks in home-based, community, and healthcare settings during the pandemic.

### SUMMARY OF THE INVENTION

One aspect of the disclosure is directed to novel aqueous povidone-iodine (PVP-I) pharmaceutical preparations. In particular, an aqueous pharmaceutical preparation of the disclosure comprises povidone-iodine (PVP-I) at a concentration of from about 0.5% to about 10% w/v and a combination of
1) alcohol ethoxylate (e.g., Pareth 25-9); and
2) one or more surfactants selected from the group of polyoxypropylene, polyoxyethylene, sodium lauryl sulfate (SLS), and poloxamer, or a combination thereof, with the aqueous pharmaceutical preparation being efficacious against one or more gram-positive bacteria (including such as, Enterococcus faecalis, Enterococcus faecium, Staphylococcus aureus, and Staphylococcus epidermidis), as demonstrated by a log reduction of the gram-positive bacteria in a value of 5 or higher after 15 seconds exposure in a standard time-kill study (e.g., that described in Example 3 or 4).

Another aspect of the disclosure provides an aqueous povidone-iodine (PVP-I) pharmaceutical preparation comprising povidone-iodine (PVP-I) at a concentration of from about 0.5% to about 10% w/v and a combination of
1) alcohol ethoxylate (e.g., Pareth 25-9); and
2) one or more surfactants selected from the group of polyoxypropylene, polyoxyethylene, sodium lauryl sulfate (SLS), and poloxamer, or a combination thereof, and optionally
3) one or more viscosity modifying agents (e.g., humectants selected from a group of celluloses);

wherein the viscosity of said aqueous pharmaceutical preparation is within the range of about 2 cps to about 500 cps, and

wherein the aqueous pharmaceutical preparation being efficacious against one or more gram-positive bacteria (including Enterococcus faecalis, Enterococcus faecium, Staphylococcus aureus, and Staphylococcus epidermidis), as demonstrated by a log reduction of the gram-positive bacteria in a value of about 4 or higher after 15 seconds exposure in a standard time-kill study (e.g., that described in Example 3 or 4).

The standard time-kill study, as mentioned herein against the bacteria, refers to an in-vitro method described in ASTM E2783-11 (2016), Standard Test Method for Assessment of Antimicrobial Activity for Water Miscible Compounds Using a Time-Kill Procedure.

In certain embodiments, the aqueous pharmaceutical preparation of the disclosure contains about 0.2% w/v of an alcohol ethoxylate (e.g., Pareth 25-9). Other embodiments provide aqueous pharmaceutical preparations containing about 0.1% w/v to about 0.5% w/v (e.g., about 0.2% w/v) of the one or more surfactants.

In one embodiment, the aqueous pharmaceutical preparation of the disclosure is a topical preparation. In another embodiment, the aqueous pharmaceutical preparation of the disclosure is a nasal spray preparation. In still another embodiment, the aqueous pharmaceutical preparation of the disclosure is a gargle preparation.

In one embodiment, the aqueous pharmaceutical preparation of the disclosure comprises a combination of alcohol ethoxylates (e.g., Pareth 25-9), and poloxamer as the one or more surfactants. Suitable poloxamers that can be used include such as poloxamer 124, poloxamer 182, poloxamer 188, poloxamer 331, and poloxamer 407, or a combination thereof.

In a separate embodiment of the disclosure, the aqueous pharmaceutical preparation comprises SLS as the one or more surfactants. In alternative embodiments, the aqueous pharmaceutical preparation contains polyoxypropylene as the one or more surfactants, for example, polyoxypropylene with a molecular mass at about 1200 to about 4000 g/mol.

In one embodiment, the disclosure provides an aqueous pharmaceutical preparation comprising about 10% w/v povidone-iodine (PVP-I), about 0.2% w/v of alcohol ethoxylates (e.g., Pareth 25-9), and about 0.2% w/v of poloxamer (e.g., poloxamer 124).

In other embodiments, the aqueous pharmaceutical preparation further comprises one or more viscosity modifying agents. Certain embodiments provide that the viscosity modifying agents are thickening agents. Other embodiments provide that the viscosity modifying agents are humectants.

One embodiment provides that the one or more viscosity modifying agents are humectants selected from the group of hydroxyethyl cellulose, hydroxypropyl methyl cellulose, and a combination thereof. As one example of the aqueous pharmaceutical preparations, the humectants are included and comprise hydroxyethyl cellulose (“HEC”). Another example provides that the humectants are included and comprise hydroxypropyl methyl cellulose (“HPMC”).

In one embodiment, the aqueous pharmaceutical preparation of the disclosure comprises about 10% w/v of PVP-I and one or more viscosity modifying agents, with the viscosity of the aqueous pharmaceutical preparation being within the range of about 10 cps to about 300 cps at 25° C. A specific embodiment provides that the one or more viscosity modifying agents comprise hydroxyethyl cellulose, and the viscosity of the aqueous pharmaceutical preparation is about 20 cps at 25° C. Another specific embodiment provides that the one or more viscosity modifying agents comprise hydroxypropyl methyl cellulose, and the viscosity of the aqueous pharmaceutical preparation is about 250 cps at 25° C.

In certain embodiments, the disclosure provides an aqueous pharmaceutical preparation comprising about 10% w/v povidone-iodine (PVP-I), about 0.2% w/v of alcohol ethoxylates (e.g., Pareth 25-9), about 0.2% w/v of poloxamer, and about 0.5% to 5.0% w/v of one or more humectants.

In a separate embodiment, the disclosure provides an aqueous pharmaceutical preparation comprising about 10% w/v povidone-iodine (PVP-I), about 0.2% w/v of alcohol ethoxylates (e.g., Pareth 25-9), about 0.2% w/v of poloxamer (e.g., poloxamer 124), and about 1.7% w/v of hydroxyethyl cellulose.

In still another embodiment, the disclosure provides an aqueous pharmaceutical preparation comprising about 10% w/v povidone-iodine (PVP-I), about 0.2% w/v of alcohol ethoxylates (e.g., Pareth 25-9), about 0.2% w/v of poloxamer (e.g., poloxamer 124), and about 1.0% w/v of hydroxypropyl methyl cellulose.

Another aspect of the present disclosure is directed to a method of reducing risk, incidence or severity of diseases (such as, COVID-19) or conditions caused by virus infections in a human subject. According to the method, an effective amount of the aqueous pharmaceutical preparation of the disclosure is topically applied to a human subject at risk, which can occur prior to, during or after the human subject is being exposed to viruses (e.g., SARS-CoV-2) or to a person infected (or suspected being infected) with the viruses.

A further aspect of the present disclosure provides a method of decreasing release of infective coronaviruses (e.g., SARS-CoV-2 viruses) from a human subject already infected with SARS-CoV-2 viruses, through topical application of an effective amount of the aqueous pharmaceutical preparation of the disclosure to the infected human subject.

Still another aspect of the disclosure provides a method of reducing the activity, viability or number of viruses (e.g., SARS-CoV-2 viruses) in the body of a human subject, who is either identified as being infected or potentially being infected with the viruses, by topically applying an effective amount of the aqueous pharmaceutical preparation of the disclosure to the human subject.

In yet another aspect, this disclosure provides a method of reducing an infection risk posed by microorganisms or viruses to a patient or a healthcare professional in a clinical setting (e.g., a surgical setting). The method comprises topically applying an antiseptically effective amount of an aqueous pharmaceutical preparation of this disclosure to the skin of the patient or the healthcare professional.

The disclosure can be understood more fully by reference to the following detailed description and illustrative examples, which are intended to exemplify non-limiting embodiments of the disclosure.

### BRIEF DESCRIPTION OF FIGURES

FIG. 1 is a table in which the results of a time-kill assay of 10% PVP-I formulation (containing poloxamer 124 and Pareth 25-9) against a variety of microorganisms are presented.

FIG. 2 is a table in which the results of a time-kill assay of placebo formulation (containing poloxamer 124 and Pareth 25-9) against a variety of microorganisms are presented.

### DETAILED DESCRIPTION OF THE INVENTION

Povidone-iodine (PVP-I) is a chemical complex of the polymer povidone (“polyvinylpyrrolidone”) and triiodide (I₃⁻). The structure of the PVP-I complex is provided as follows:

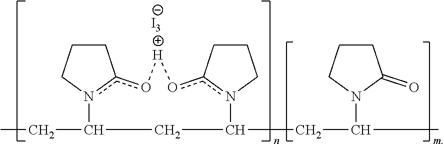

Povidone-iodine is a form of an iodophor. An iodophor is a preparation containing iodine complexed with a solubilizing agent, e.g., a surfactant or water-soluble polymers (in this case, povidone). When in solution, povidone-iodine complex releases free iodine.

One aspect of the present disclosure provides an aqueous pharmaceutical preparation comprising povidone-iodine (PVP-I) at a concentration of from about 0.5% to about 10% w/v and a combination of
1) alcohol ethoxylate (e.g., Pareth 25-9); and
2) one or more surfactants selected from the group of polyoxypropylene, polyoxyethylene, sodium lauryl sulfate (SLS), and poloxamer, or a combination thereof, wherein the aqueous pharmaceutical preparation has efficacy against one or more gram-positive bacteria selected from the group of Enterococcus faecalis, Enterococcus faecium, Staphylococcus aureus, and Staphylococcus epidermidis, with the efficacy being demonstrated by a log reduction of the one or more gram-positive bacteria in a value of 5 or higher after 15 seconds exposure in a standard time-kill study (e.g., that described below in Example 3 or 4). In some embodiments, the aqueous pharmaceutical preparation of the disclosure is efficacious against one or more gram-positive bacteria selected from the group of Enterococcus faecalis, Enterococcus faecium, Staphylococcus aureus, and Staphylococcus epidermidis, with the efficacy being demonstrated by a log reduction of the gram-positive bacteria in a value of about 4 or higher after 15 seconds exposure in a standard time-kill study.

The term “pharmaceutical preparation” as used in this disclosure refers to a material that may be used in medical fields and/or as consumer products (e.g., cleansing products). The material can be used neat (e.g., as supplied) or after dilution. The term “pharmaceutical” used herein relates to one of the functions of the material, that is, the material may protect healthcare professionals and/or general publics from potentially serious diseases or health conditions caused by microbial infections. In certain embodiments of this disclosure, the term “pharmaceutical preparation” and the term “pharmaceutical formulation” are used interchangeably.

The term “effective amount” refers to an amount sufficient to deliver efficacy, wherein the efficacy is achieved through reduction or elimination of target microorganisms within a specified amount of time.

Alcohol ethoxylates (AEs) that can be used in the pharmaceutical preparations of the disclosure are generally suitable for human uses. These alcohol ethoxylates are not observed to be mutagenic, carcinogenic, or skin sensitizers, nor cause reproductive or developmental effects. Alcohol ethoxylates are in the following formula (Formula A):

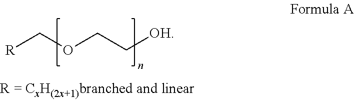

Suitable alcohol ethoxylates include, for example, C12-15 alcohol ethoxylate (hereinafter “Pareth 25-9”) x=12-15 in Formula A; with CAS No. 68131-39-5); C10-12 pareth-3 (CAS No. 66455-15-0); C1115 pareth-3 (CAS No. 68131-40-8); C11-15 pareth-7 carboxylic acid (CAS No. 68954-90-5); C11-21 pareth-3 (CAS No. 246538-82-9); C12-13 pareth-10 phosphate (CAS No. 68130-45-0); C12-13 pareth-5 carboxylic acid CAS No. 70750-17-3); C12-13 pareth-6 (CAS No. 66455-14-9); C12-14 pareth (CAS No. 68439-50-9); C12-15 pareth-2 phosphate (CAS No. 149919-05-1); C12-15 pareth-7 carboxylic acid (CAS No. 88497-58-9); C12-15 pareth-9 hydrogenated tallowate (CAS No. 246538-68-1); C12-16 pareth-1 (CAS No. 68551-12-2); C12-18 pareth-14 (CAS No. 68213-23-0); C14-15 pareth-2 (CAS No. 68951-67-7); C14-15 pareth-8 carboxylic acid (CAS No. 119147-75-0) (all the chemical specifics can be retrieved through the product website of Parchem^{™} fine & specialty chemicals; assessed in May 2021).

One embodiment provides that the alcohol ethoxylate in the present pharmaceutical preparation is Pareth 25-9. Another embodiment provides that the alcohol ethoxylate in the present pharmaceutical preparation is a combination of Pareth 25-9 and one or more of other alcohol ethoxylates.

In certain embodiments, the alcohol ethoxylates are used as surfactants in the present pharmaceutical preparation. In another embodiment, the alcohol ethoxylates are used as emulsifiers in the present pharmaceutical preparation. In still another embodiment, the alcohol ethoxylates are used as dispersants in the present pharmaceutical preparation. In other embodiments, the alcohol ethoxylates are used as stabilizers in the present pharmaceutical preparation.

Polyoxypropylene (or polypropylene glycol; “PG”) is a general polymer class of which propylene glycol is a member.

Polyoxyethylene (or polyethylene glycol; “POE” or “PEG”) is a polymer of ethylene glycol having a general formula HO—(CH₂CH₂O)ₙ—H. Polyoxyethylenes have been used medically and also been incorporated into a variety of consumer products (e.g., skin creams, toothpastes, food and drinks). Suitable polyoxyethylenes for the aqueous pharmaceutical preparations of the disclosure include, such as, polyoxypropylene with a molecular mass at about 1200 to about 4000 g/mol (e.g., PEG300).

In certain embodiments, the aqueous pharmaceutical preparation of the disclosure contains a combination of alcohol ethoxylate (e.g., Pareth 25-9) and polyoxypropylene. One instance provides that the polyoxypropylene has a molecular mass at about 1200 to about 4000 g/mol. In separate embodiments, the polyoxypropylene comprises a content of polyoxyethylene ranging from about 40 to about 70%.

Sodium lauryl sulfate (or sodium dodecyl sulfate; “SLS”) has the chemical formula of CH₃(CH₂)₁₁SO₄Na, which is an organosulfate salt. It is an anionic surfactant, and has been used in many domestic cleaning, personal hygiene and cosmetic, medical, and food products.

In separate embodiments, the aqueous pharmaceutical preparation of the disclosure contains a combination of alcohol ethoxylates (e. g., Pareth 25-9) and SLS.

In certain embodiments, the aqueous pharmaceutical preparation of the disclosure contains a combination of an alcohol ethoxylate (e.g., Pareth 25-9) and a poloxamer.

Poloxamers are nonionic triblock copolymers composed of a central hydrophobic chain of polyoxypropylene (poly(propylene oxide)) flanked by two hydrophilic chains of polyoxyethylene (poly(ethylene oxide)), in the following general structure:

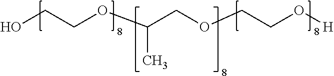

with a=2-130 and b=15-67

As shown in the above general structure of the poloxamer, the middle block is hydrophobic and the two end blocks are hydrophilic, the poloxamer behaves as polymer surfactant. It is generally used as nonionic polymer surfactant. Poloxamer can also function as antifoaming agents, wetting agents, dispersants, thickeners, and emulsifiers, and the like.

Poloxamer, which can be used to formulate the present aqueous pharmaceutical preparation, includes such as, poloxamer 124, poloxamer 182, poloxamer 188, poloxamer 331, and poloxamer 407, or a combination thereof. In one embodiment, the aqueous pharmaceutical preparation comprises poloxamer 124 or poloxamer 407, or a combination thereof. One example provides that the pharmaceutical preparation contains a poloxamer, which is poloxamer 124.

As an example of the aqueous pharmaceutical preparations of the disclosure, the preparation contains about 0.2% w/v of the alcohol ethoxylate (e.g., Pareth 25-9). In such an aqueous pharmaceutical preparation, the preparation may further comprise about 0.1% w/v to about 0.5% w/v (e.g., about 0.2% w/v) of the one or more surfactants.

As one specific example of the aqueous pharmaceutical preparation, the preparation contains about 10% w/v povidone-iodine (PVP-I), about 0.2% w/v of an alcohol ethoxylate (e. Pareth 25-9), and about 0.2% w/v of poloxamer (e.g., poloxamer 124).

In certain embodiments, the aqueous pharmaceutical preparation of this disclosure is a topical preparation (e.g., topical solutions, and surgical scrubs). One example of the topical preparation comprises about 10% w/v povidone-iodine (PVP-I), about 0.2% w/v of alcohol ethoxylates (e.g., Pareth 25-9), about 0.2% w/v of poloxamer (e.g., poloxamer 124), and about 1.7% w/v of hydroxyethyl cellulose. Another example of the topical preparation comprises about 10% w/v povidone-iodine (PVP-I), about 0.2% w/v of alcohol ethoxylates (e.g., Pareth 25-9), about 0.2% w/v of poloxamer (e.g., poloxamer 124), and about 1.0% w/v of hydroxypropyl methyl cellulose.

In certain embodiments, the aqueous pharmaceutical preparation is a gargle preparation. In certain embodiments, the gargle preparation contains about 0.5% to 2.0% w/v povidone-iodine (PVP-I). One example relates to a 0.5% w/v PVP-I gargle preparation. In another example, the disclosure provides a 1.0% w/v PVP-I gargle preparation. The gargle preparation can also contain 1.25% w/v PVP-I.

In one embodiment, the aqueous pharmaceutical preparation is an oral rinse. Certain embodiments provide that the oral rinse contains about 0.5% to 2.0% w/v povidone-iodine (PVP-I). One example is a 0.5% w/v PVP-I oral rinse. As another example, the disclosure provides a 1.0% w/v PVP-I oral rinse. One embodiment relates to a 1.25% w/v PVP-I oral rinse.

In separate embodiments, the aqueous pharmaceutical preparation is a nasal spray preparation. In one embodiment, the nasal spray preparation of the disclosure contains about 0.5% to about 5.0% w/v povidone-iodine (PVP-I). One example provides a 0.5% w/v PVP-I nasal spray preparation. In other instances, this disclosure relates to a 1.0% w/v, 2.0% w/v, or 2.5% w/v PVP-I nasal spray preparation.

Depending on how the aqueous pharmaceutical preparation of this disclosure is formulated, the PVP-I can be provided in a pharmaceutically acceptable carrier, which can further include a variety of excipients. For example, a pharmaceutically acceptable carrier can include one or more of a pH adjusting agent, a solubilizing agent, a tonicity or isotonicity adjusting agent, a viscosity modifying agent, a suspension stabilizer, a preservative, the like, and combinations thereof (see also below on the “Topical Preparations” for additional excipients).

The aqueous pharmaceutical preparation of this disclosure can further comprise a pH adjusting agent. The addition of a pH-adjusting agent is sometimes preferred when the pharmaceutical preparations are applied topically on the skin of an animal (e.g., a human subject). The pH of these treated areas is typically lower than 6.0. Hence, in certain embodiments, the aqueous pharmaceutical preparations of the disclosure have a pH value of between about 3 and about 6, thereby avoiding irritations to the skin of a human subject. In one embodiment, the aqueous pharmaceutical preparations of this disclosure have a pH value of between about 3.5 and about 4.5. Certain embodiments provide the aqueous pharmaceutical preparations having a pH value of between about 4.0 and about 4.2.

Suitable pH adjusting agents include, for example, sodium hydroxide, adipic acids, acetic acid, glycines, citric acid, hydrochloric acid, phosphoric acid, disodium hydrogen phosphate, calcium hydroxide, potassium hydroxide, magnesium aluminometasilicate, the like, and buffers or any combinations thereof. In certain embodiments, a citrate/phosphate buffer in combination of sodium hydroxide is used to establish and/or maintain a desired pH value of an aqueous pharmaceutical preparations of this disclosure. A citrate/phosphate buffer, in one embodiment, has a pH value of about 4, which is used to prepare the pharmaceutical preparation of the disclosure. Such a citrate/phosphate buffer can be prepared through mixing citric acid and disodium hydrogen phosphate in water.

The aqueous pharmaceutical preparation of this disclosure can also include a viscosity modifying agent. In certain embodiments, the viscosity modifying agents are thickening agents. In other embodiments, the aqueous pharmaceutical preparations of this disclosure comprise one or more humectants as the viscosity modifying agents.

Humectants are hygroscopic substances used to keep things moist. These substances can attract water from the air or from deeper in the skin. Humectants exist in many different forms, such as, natural, synthetic, and naturally derived. Humectants suitable for the aqueous pharmaceutical preparations of this disclosure include, such as, celluloses (e.g., hydroxyethyl cellulose, and hydroxypropyl methyl cellulose), glycols (e.g., propylene glycol, hexylene glycol, and butylene glycol), alpha hydroxy acids (e.g., lactic acid), polymeric polyols (e.g., polydextrose), lithium chloride, glyceryl triacetate, sugar alcohols (e.g., glycerol, sorbitol, xylitol, and maltitol) and sodium hexametaphosphate.

Also suitable viscosity modifying agent can be selected according to the knowledge of a person with ordinary skills in the relevant arts (e.g., pharmaceutical, cosmetic, or consumer product art), which may include, such as, one or more from the group of acacia, agar, alginic acid, aluminum monostearate, ammonium alginate, attapulgite, bentonite, calcium alginate, calcium lactate, carbomer, carboxymethylcellulose calcium, carboxymethylcellulose sodium, carrageenan, cellulose, ceratonia, ceresin, cetostearyl alcohol, cetyl palmitate, chitosan, colloidal silicon dioxide, corn syrup solids, cyclomethicone, ethylcellulose, gelatin, glyceryl behenate, guar gum, hectorite, hydrophobic colloidal silica, hydroxyethyl cellulose, hydroxyethylmethyl cellulose, hydroxypropyl cellulose, hydroxypropyl starch, hypromellose, magnesium aluminum silicate, maltodextrin, methylcellulose, myristyl alcohol, octyldodecanol, palm oil, pectin, polycarbophil, polydextrose, polyethylene oxide, polyoxyethylene alkyl ethers, polyvinyl alcohol, potassium alginate, propylene glycol alginate, pullulan, saponite, sodium alginate, starch, sucrose, sugar, sulfobutylether beta-cyclodextrin, tragacanth, trehalose, and xanthan gum.

The present inventors became aware that there has been an unmet need in surgical settings for an antiseptic solution with less potential to drip once applied to the skin and minimizes pooling of the solution. Pooling and collection of antiseptics solutions can lead to potential irritation of skin after being exposed for a long period of time, which is a common scenario in a surgical environment. Accordingly, the viscosity of the aqueous pharmaceutical preparations of this disclosure has been carefully adjusted to fulfill the need in the industry for such an improved antiseptic solution.

The viscosity of the aqueous pharmaceutical preparation of this disclosure can be in the range between about 2 centipoise (“cps”) to about 500 cps at 25° C., or in the range between about 10 cps to about 300 cps at 25° C. In certain embodiments, an aqueous pharmaceutical preparation of this disclosure has a viscosity within the range between about 150 cps to about 350 cps at 25° C., or within the range of about 200 cps to about 300 cps at 25° C. In one instance, an aqueous pharmaceutical preparation of this disclosure has a viscosity value of about 250 cps at 25° C.

In other embodiments, the viscosity of an aqueous pharmaceutical preparation of this disclosure is within the range between about 2 cps to about 100 cps at 25° C. In one embodiment, an aqueous pharmaceutical preparation of this disclosure has a viscosity within the range between about 10 cps to about 30 cps at 25° C. One example of the aqueous pharmaceutical preparations has a viscosity value of about 20 cps at 25° C.

Another embodiment provides that the aqueous pharmaceutical preparation has the viscosity between 5 cps and 10 cps at 25° C. In certain embodiments, the aqueous pharmaceutical preparation of this disclosure has the viscosity between 6 cps and 7.5 cps at 25° C.

The viscosity is measured on a 5 wt. % aqueous solution using a model RVF Brookfield viscosimeter (spindle no. 2/rotational speed 2 rpm), of 400 to 4000 cB, measured on a 2 wt. % aqueous Solution using the stated viscosimeter (spindle no. 1 or 3/rotational speed 10 rpm) or of 1650 to 10000 cB, measured on a 1 wt. % aqueous solution using the stated viscosimeter (spindle no. 2/rotational speed 2 rpm).

In certain embodiments, the viscosity achieved by the aqueous pharmaceutical preparations of this disclosure allows ease of spreadability across the skin, while minimizing dripping and pooling of the solution once applied topically.

Further, the aqueous pharmaceutical preparation may include suspension stabilizers such as, substituted celluloses (e.g. methylcellulose, hydroxypropylmethylcellulose), polyvinyl alcohol, polyvinylpyrrolidone. Preservatives (e.g. chlorocresol, phenylmercury compounds, phenylethanol, benzalkonium chloride or mixtures thereof) and isotonicity adjusting agents (e.g., sodium chloride) may also be included.

Non-limiting examples of solubilizing agents can include phosphate-buffered saline (PBS), Dulbecco's PBS, Alsever's solution, Tris-buffered saline (TBS), water, balanced salt solutions (BSS), such as Hank's BSS, Earle's BSS, Grey's BSS, Puck's BSS, Simm's BSS, Tyrode's BSS, BSS Plus, Ringer's lactate solution, normal saline, the like, or combinations thereof. Solubilizing agents can be present in the pharmaceutically acceptable carrier in various amounts depending on the particular pharmaceutical preparations, method of use, and etc.

Non-limiting examples of tonicity agents can include the solubilizing agents previously listed, as well as sodium chloride, potassium chloride, calcium chloride, magnesium chloride, mannitol, sorbitol, dextrose, glycerin, propylene glycol, ethanol, trehalose, the like, or combinations thereof. The tonicity agent can be used to provide an appropriate tonicity of the formulation. Tonicity agents can be present in the pharmaceutically acceptable carrier in various amounts depending on the particular pharmaceutical preparations, methods of use, and etc.

### Topical Preparations

In one aspect, the aqueous pharmaceutical preparation of the disclosure is a topical preparation. For the purpose of this disclosure, a topical preparation is a preparation or medication that is applied to a particular place on or in the body of an animal (e.g., a human subject).

In certain embodiments, the aqueous pharmaceutical preparation is in the form of a solution, which can be applied as, for example, scrub, rinse, drop, spray, or foam. Topical solutions of this disclosure are generally of a low viscosity, and use water as the base solvent. In one embodiment, the topical solutions of this disclosure may also include alcohol or other appropriate co-solvents.

The PVP-I topical solutions of this disclosure release iodine when applied to skin. In one embodiment, the topical solution of this disclosure is a surgical scrub. In one embodiment, the topical solution of this disclosure is a spray. In one embodiment, the topical solution of this disclosure is used in surgical or healthcare settings. In another embodiment, the topical solution of this disclosure is used as a consumer antiseptic solution.

In another aspect, the aqueous pharmaceutical preparation of the disclosure can also be in a form of ointments (e.g., solution ointment, suspension ointment), creams, gels, and topical suspensions, all of which use water as one of their base solvents.

Creams of this disclosure may include oil-in-water or water-in-oil emulsions. Chiefly used for the oily phase are fatty alcohols (e.g. lauryl, cetyl or stearyl alcohol), fatty acids (e.g. palmitic or stearic acid), liquid or solid paraffins or ozokerite, liquid to solid waxes, natural or partially synthetic fat, hardened oils, or fatty acid partial esters of glycerol. Suitable emulsifiers can also be present, include such as, nonionic surfactants (e.g. fatty acid esters of polyalcohols or ethylene oxide adducts thereof) or anionic surfactants (such as, alkali metal salts of fatty alcohol sulphates). It is also possible to add to the aqueous phase inter alia agents which prevent the cream drying out, e.g. polyalcohols such as glycerol, sorbitol, propylene glycol and/or polyethylene glycols, also preservatives, fragrances etc.

Ointments of this disclosure include such as, hydrous ointments (aka “oily creams”). It may be made from a mixture of wool alcohols ointments (which contains lanolin, a waxy substance obtained from wool of animals, e.g., sheep) and water. In certain embodiments, an antimicrobial preservative (e.g., phenoxyethanol) is included.

Other antimicrobial agents and preservatives may be included in the aqueous pharmaceutical preparations of this disclosure, as long as they are compatible with the pharmaceutical preparations suitable for topical applications.

The aqueous pharmaceutical preparations may include other solvents (either in trace amount or used as a co-solvent), such as, acetonitrile, alcohols (e.g., ethanol, isopropyl alcohol), ethyl acetate, dimethylsulfoxide (DMSO), dimethylformamide (DMF), ketones (e.g., acetone), heptane, tetrahydrofuran (THF), ethers (e.g., ethyl ether), polyethylene glycol, glycerol, and the like.

In certain embodiments, the aqueous pharmaceutical preparations are stable at room temperature (about 20-25° C.) for at least 6 months, 12 months, 18 months, 24 months, 36 months, or 48 months. “Stability”, in this disclosure, is defined as where the final PVP-I concentration is at least 85% of the labeled concentration.

In certain embodiments, the topical preparations of this disclosure are particularly suitable for use as antiseptic solutions in clinical settings (e.g., surgical settings). The viscosity of these topical preparations allows ease of spreadability across the skin, while minimizing dripping and pooling of the solution once applied topically.

### Efficacy Against Microorganisms and Viruses

PVP-I has well-established general antimicrobial activity, demonstrating in vitro efficacy against gram-positive, gram-negative and some spore-forming bacteria (clostridia, Bacillus spp.) and mycobacteria and a wide range of enveloped and nonenveloped viruses (Eggers et al., Infect Dis. Ther, 2018, 7:249-259).

In certain embodiments, the aqueous pharmaceutical preparations of this disclosure demonstrate a strong bactericidal and virucidal efficacy against a variety of microorganisms, such as gram-negative and gram-positive bacteria, bacterial spores, fungi, protozoa, and viruses, yeasts. In certain embodiments, the aqueous pharmaceutical preparations of this disclosure have efficacy against specific bacteria and yeasts, including such as, Arinetobacter baumannii (ATCC #19606); Bacteroides fragilis (ATCC #25285); Candida albicans (ATCC #10231); Enterobacter sped es (ATCC #13048); Enterococcus faecalis (ATCC #29212); Enterococcus faecalis VRE, MDR (ATCC #51575); Enterococcus faecium VRE (ATCC #700221), Escherichia coli (ATCC #11229); Escherichia coli (ATCC #25922); Haemophilus influenzae (ATCC #19418), Klebsiella pneumoniae (ATCC #13883); Klebsiella pneumoniae (ATCC #27736); Micrococcus yunnanensis (ATCC #7468, formerly known as Micrococcus luteus); Proteus mirabilis (ATCC 47002); Pseudomonas aeruginosa (ATCC #15442); Pseudomonas aeruginosa (ATCC #27853); Serratia marcescens (ATCC 414756); Staphylococcus aureus (ATCC 46538); Staphylococcus aureus (ATCC #29213); Staphylococcus epidermidis (ATCC #12228); Staphylococcus epidermidis MRSE (ATCC #51625); Staphylococcus haemolyticus (ATCC #29970); Staphylococcus hominis (ATCC #27845); Staphylococcus saprophyticus (ATCC #15305); Streptococcus pneumoniae (ATCC #6303); Streptococcus pneumoniae (ATCC #49619); Streptococcus pyogenes (ATCC #14289); Streptococcus pyogenes (ATCC #19615); Burkholderia cepacia (ATCC 2541); Burkholderia cenocepacia (ATCC BAA-245); Burkholderia multivorans (ATCC BAA-247); Campylobacter jejuni (ATCC #33291), Enterococcus faecalis (ATCC #19433); Escherichia coli 0157117 (ATCC #35150); Salmonella enteriditis (ATCC #13074 Salmonella enterica (ATCC #14028); Shigella sonnei (ATCC #25931); Listeria monocytogenes (ATCC #19115); Staphyloccus aureus, MRSA (ATCC #33591); Candida albicans (ATCC #10231); Candida tropicalis (ATCC #750), and the like; etc.

Certain embodiments provide that the aqueous pharmaceutical preparation of the disclosure is particularly efficacious against certain gram-positive bacteria including such as, Enterococcus faecalis, Enterococcus faecium, Staphylococcus aureus, and Staphylococcus epidermidis, as demonstrated by a log reduction in a value of 5 or higher 15 seconds after these bacteria are being exposed in a time-kill study (as presented in Example 4). Another embodiment provides that the aqueous pharmaceutical preparations of the disclosure achieve greater than 6-log reduction across all the four gram-positive bacteria (i.e., Enterococcus faecalis, Enterococcus faecium, Staphylococcus aureus, and Staphylococcus epidermidis) after 15 seconds in the time-kill study. In a separate embodiment, the aqueous pharmaceutical preparations of the disclosure achieve greater than 6-log reduction in at least three of the four gram-positive bacteria (i.e., Enterococcus faecalis, Enterococcus faecium, Staphylococcus aureus, and Staphylococcus epidermidis) after 15 seconds in the time-kill study. In another embodiment, the aqueous pharmaceutical preparations of the disclosure achieve greater than 6-log reduction in at least two of the four gram-positive bacteria after 15 seconds in the time-kill study. In still another embodiment, the aqueous pharmaceutic& preparations of the disclosure achieve greater than 6-log reduction in at least one of the four gram-positive bacteria after 15 seconds in the time-kill study.

In another embodiment, the aqueous pharmaceutical preparations of the disclosure achieve greater than 6-log reduction in at least two of the four gram-positive bacteria after 15 seconds in the time-kill study. In still another embodiment, the aqueous pharmaceutical preparations achieve greater than 6-log reduction in all the four gram-positive bacteria after 15 seconds in the time-kill study.

In separate embodiments, the aqueous pharmaceutical preparations of the disclosure demonstrate strong virucidal efficacy against a variety of viruses (e.g., coronaviruses), including such as Middle East Respiratory Syndrome coronavirus (ERS), severe acute respiratory syndrome coronavirus (SARS), severe acute respiratory syndrome coronavirus 2 (SARS-CoV-2 Ebola virus, and influenza A virus.

In certain embodiments, the aqueous pharmaceutical preparations of the disclosure demonstrate strong and rapid virucidal efficacy against coronaviruses, demonstrated by achieving greater than a 3-log reduction of the viruses within 5 minutes or less after the viruses are exposed (or incubated) in the time-kill study. In certain embodiments, the aqueous pharmaceutical preparations achieve greater than a 3-log reduction of the viruses within 3 minutes, 2 minutes, 60 seconds, 45 seconds, 30 seconds, or 15 seconds after the viruses are exposed (or incubated) in the time-kill study. In one embodiment, the aqueous pharmaceutical preparations achieve greater than a 3-log reduction of the viruses at all timepoints between 5 minutes and 15 seconds (endpoints included) after the viruses are exposed (or incubated) in the time-kill study.

Other embodiments of the disclosure provide that the aqueous pharmaceutical preparations achieve greater than a 4-log reduction of the coronaviruses within 5 minutes or less (e.g., within 3 minutes, 2 minutes, 60 seconds, 45 seconds, 30 seconds, or 15 seconds) after the coronaviruses are exposed (or incubated) in the time-kill study.

In one embodiment, the coronavirus is SARS. In another embodiment, the coronavirus is SARS-CoV-2.

One example provides that the aqueous pharmaceutical preparation achieves about 4-log reduction of SARS-CoV-2 viruses 15 seconds after the SARS-COV-2 viruses are exposed in a time-kill study.

The time-kill study against coronaviruses can use either human or animal coronavirus strains (e.g., murine coronavirus strains). Sometimes, surrogate test viruses for coronaviruses (e.g., SARS-CoV-2) can also be used. For example, suitable human viral strains that can serve as surrogates for SARS-CoV-2 including such as, coronavirus strain 0C43 (ZeptoMetrix Corp. #0810024CF), coronavirus strain NL63 (ZeptoMetrix Corp. #0810228CF), and coronavirus strain 229E (ATCC® VR-740^{™}).

In certain embodiments, an aqueous pharmaceutical preparations of the disclosure demonstrates a strong and rapid efficacy against the four gram-positive bacteria (i.e., Enterococcus faecalis, Enterococcus faecium, Staphylococcus aureus, and Staphylococcus epidermidis), shown by achieving greater than 6-log reduction of each of the bacteria after 15 seconds in a time-kill study, while the aqueous pharmaceutical preparation also demonstrates a strong and rapid virucidal efficacy against coronaviruses (e.g., SARS-CoV-2 viruses), demonstrated by achieving greater than a 3-log reduction of the coronaviruses in 5 minutes or less (e.g., within 60 seconds, within 45 seconds, within 30 seconds, or within 15 seconds) after the viruses are exposed in a time-kill study. One embodiment provides that a 4-log reduction of SARS-CoV-2 viruses is achieved in 15 seconds post exposure.

### Method of Uses

One aspect of this disclosure is directed to methods of reducing risk, incidence, or severity of diseases or conditions caused by virus infection in an animal (e.g., a human subject In accordance to these methods, an aqueous pharmaceutical preparation of the disclosure is topically applied to the animal (e.g., a human subject) being directly exposed to viruses. Alternatively, the methods of the disclosure are applicable when the animal (e.g., a human subject) is being exposed (or in close physical proximity, e.g., within 12 feet or 6 feet) to another animal (e.g., a person) who is infected (or suspected being infected) with the viruses. The methods of this disclosure are efficacious despite that the aqueous pharmaceutical preparation is applied prior to, during, or after the exposure.

In certain embodiments, the methods involve applying an aqueous pharmaceutical preparation of this disclosure to a human subject, before the human subject is being exposed either to the viruses or a person infected (or suspected of being infected) with the viruses. In another embodiment, the methods involve applying an aqueous pharmaceutical preparation of this disclosure to a human subject after being exposed to either the viruses or a person infected (or suspected of being infected) with the viruses.

In another aspect, this disclosure provides a method of decreasing release of viruses from an animal (e.g., a human subject) already infected with viruses, thus reducing the incidence of secondary infections caused to others (e.g., human beings). The method involves topically applying to the infected animal (e.g., the human subject) an effective amount of an aqueous pharmaceutical preparation of the disclosure.

This disclosure also provides a method of reducing the activity, viability or number of the viruses in the body of an animal (e.g., a human subject), who is identified as being infected or potentially being infected with the viruses. According to this method, an aqueous pharmaceutical preparation of this disclosure is topically applied to such an animal (e.g., a human subject).

A topical application means that a pharmaceutical preparation (or a medication) is applied to a particular place on or in the body of an animal (e.g., a human subject). In certain embodiments, the methods of this disclosure comprise a step of topically applying the aqueous pharmaceutical preparation to the skin of the human subject. For example, the aqueous pharmaceutical preparation of this disclosure can be topically applied to a human subject's hands, face, torso skin, arms, legs, feet, and hips etc. The skin area for application can be either intact or damaged.

When applied to the skin of a human subject, the aqueous pharmaceutical preparation of this disclosure may be used in conjunction with other skin treatment material or ingredients, such as but not limited to, sunscreen, vitamins, plant extracts, and moisturizers.

In another embodiment, the methods involve applying an aqueous pharmaceutical preparation (e.g., in the form of drops or spray, and the like) of this disclosure to a nasal passage of a human subject. Nasal passages are known to provide effective entrapment mechanism to prevent particles and aerosols (including microbes) from being further transported along the airways to the bronchioles and lungs, which may result in accumulation of viruses and bacteria in in the nasal passages. In certain embodiments, the disclosure provides a method for inhibiting proliferation of viruses or bacteria within the nasal passages of an animal (e.g., a human subject). In other embodiments, the disclosure provides a method for reducing the amount or frequency of viruses or bacteria shredding from the nasal passages of an infected animal (e.g., a human subject).

In certain embodiments, the methods of this disclosure involve applying an aqueous pharmaceutical preparation to the mouth of a human subject as a gargle preparation or an oral rinse (e.g., a mouthwash). According to the methods of this disclosure, the human subject may swish the \preparation for a short time (e.g., from 5 seconds to 5 minutes) in the mouth and spit it out. In one embodiment, the human subject swishes the preparation for a period of time between about 15 seconds to about 60 seconds before spitting it out. In other embodiments, the human subject swishes the gargle preparation for about 15 seconds, 30 seconds, 45 seconds, or 60 seconds before spitting it out.

In certain embodiments, the viruses that may cause infection symptoms or even diseases in an animal (e.g., a human being) are highly pathogenic viruses. In certain embodiments, the viruses are coronaviruses, including such as, SARS-CoV2, SARS, and MERS viruses. In one specific embodiment, the viruses are SARS-CoV2 viruses.

This disclosure also provides a method for reducing in a human subject the risk of infection or transmission of infection caused by coronaviruses (e.g., SARS-CoV-2) when exposed to coronaviruses (either airborne viruses or objects contaminated with viruses) or animals (e.g., human beings) infected (or suspected being infected) with the coronaviruses. The methods involve topically applying an effective amount of the aqueous pharmaceutical preparations of this disclosure to the human subject.

All the afore-mentioned methods can be utilized as frequently as deemed appropriate by an ordinarily skilled healthcare professional or in accordance with the common knowledge in regards to an antiseptic product of its nature. In certain embodiments, the afore-mentioned methods involve topically applying an aqueous pharmaceutical preparation of this disclosure with a frequency of between 1 and 24 times daily, which either consecutively or intermittently last for a duration of up to 30 days, including such as, 28 days, 21 days, 14 days, 7 days, 5 days, 3 days or 1 day.

The human subjects as afore mentioned can include, such as, healthcare workers, first medical responders, law enforcement officers, teachers, and general consumers.

### Examples

### Example 1. Preparation of 10% PVP-I formulation of the Disclosure

A solution of 10% PVP-I formulation in a batch size of 20 L was prepared. Components used to formulate the solution are provided in Table 1:

TABLE 1

| Components | % w/v | Batch (g) |
|---|---|---|
| PVP-I | 10.0 | PVPI Amt. |
| Pareth 25-9 | 0.2 | 40 |
| Poloxamer 124 | 0.2 | 40 |
| ION Sodium hydroxide | To adjust pH | |
| Citrate/phosphate buffer | −89.4 | q.s. to 20 L |

The citrate/phosphate buffer in Table 1 was prepared by the following procedure:
a) adding citric acid (258.3 g, MW=210.14 g/mol) to 20 L water;
b) mixing the resulting mixture from step a) until all solids were completely dissolved;
c) adding disodium hydrogen phosphate (218.9 g; MW=141.96 g/mol) and mixing for sufficient time until all solids were completely dissolved; and
d) obtained a citrate/phosphate buffer with a pH value between 3.9 and 4.1.

The 10% PVP-I formulation was prepared according to the following process:
1) A 15 L of citrate/phosphate buffer was added to a carboy, mixed (by a mixer) to obtain a vortex;
2) 40 g Pareth 25-9 was added to the carboy, followed by the addition of 40 g Poloxamer 124;
3) the mixture resulted from step 2) was mixed until all solids were completely dissolved;
4) PVP-I was subsequently added and was mixed for another 1 to 2 hours until all solids were completely dissolved;
5) Samples were obtained from step 4) to test for pH value;
6) According to the sample pH value obtained in step 5), 10 N NaOH was used to adjust the PVP-I solution in step 4) to a value between 4.0 to 4.2; and
7) q. s. to reach a final volume of 20 L by adding the citrate/phosphate buffer, wherein the final solution pH was measured.

The resulting 10% PVP-I formulation from the above process was stored into induction-sealed HDPE bottles and kept at room temperature (20-25° C.).

### Example 2: Topical Preparations of the Disclosure

Two more topical preparations (containing about 10.0% w/v of PVP-I), in a batch size of 20 L each, were prepared according to the process as described in Example 1. Components used to formulate these two topical preparations are provided in Tables 2 and 3, respectively:

TABLE 2

| Components | % w/v | Batch (g) |
|---|---|---|
| PVP-I | 10.0 | PVPI Amt. |
| Pareth 25-9 | 0.2 | 40 |
| Poloxamer 124 | 0.2 | 40 |
| Hydroxyethyl cellulose | 1.7 | 340 |
| ION Sodium hydroxide | To ac | just pH |
| Citrate/phosphate buffer | −87.9 | q.s. to 20 L |

The resulting topical preparation of Table 2 (also called “HEC formulation”) has a viscosity value of about 20 cps.

TABLE 3

| Components | % w/v | Batch (g) |
|---|---|---|
| PVP-I | 10.0 | PVPI Amt. |
| Pareth 25-9 | 0.2 | 40 |
| Poloxamer 124 | 0.2 | 40 |
| Hydroxypropyl methyl cellulose | 1 | 200 |
| ION Sodium hydroxide | To ac | just pH |
| Citrate/phosphate buffer | −88.6 | q.s. to 20 L |

The resulting topical preparation of Table 3 (also called “HPMC formulation”) has a viscosity value of about 250 cps.

### Example 3. Time-Kill Efficacy Studies Against Bacteria and Yeasts

The procedure was in accordance with ASTM E2783-11, Standard Test Method For Assessment of Antimicrobial Activity for Water Miscible Compounds Using a Time-Kill Procedure (protocol that can be retrieved from https://www.astm.org/Standards/E2783.htm).

### Reagents:

Agar was melted prior to test and cooled to 45° C.±1° C., depending on organism: Tryptone Soya Agar (TSA) for aerobic bacteria; Malt Extract Agar (MEA) for yeasts; Brain Heart Infusion Agar (BHI) for Streptococci; Chocolate Agar for H. influenzae; and TSA+5% sheep blood for C. jejuni.

Diluent depended on organism: Tryptone Sodium Chloride Solution for bacteria and yeasts. Aliquoted into tubes to allow the preparation of organism suspensions and viable count serial dilutions. Neutralizer included lecithin 3 g/1; polysorbate 80, 30 g/1; sodium thiosulphate 5 g/1; L-histidine 1 g/l in Tryptone Sodium Chloride Solution.

### Test Materials:

Test formulations: 1) 5.0% PVP-I antiseptic spray (i.e., Betadine® spray); 2) 10.0% PVP-I antiseptic solution; 3) 10% PVP-I (Poloxamer 124/Pareth 25-9); and 4) Placebo (Poloxamer 124/Pareth 25-9).

### Material Preparation:

Controls were included in time-kill assays for all strains.

Experimental control verified absence of a lethal effect of the test method conditions. Experimental control was prepared by dispensing a 9.9 ml volume of sterile distilled water into a tube. A 0.1 ml volume of Nv was added, mixed and a stopwatch immediately started. After the longest chosen contact time had elapsed, 1.0 ml in duplicate was plated using either the pour or spread plate method.

Neutralizer toxicity control verified absence of toxicity of the neutralizer to the organism. This control was prepared by dispensing a 9.0 ml volume of the chosen neutralizer into a tube. A 1.0 ml of Nv-1 was then added, mixed and a stopwatch immediately started. After 5 minutes neutralization time, 1.0 ml in duplicate was plated using either the pour or spread plate method.

Neutralizer validation control verified the efficacy of the neutralizer in inactivating the antimicrobial agent after the contact time, which was prepared by dispensing a 9.9 ml volume of sample under test into a tube. 2.7.4.3. A 0.1 ml volume of diluent was added, mixed and a stopwatch immediately started. After the longest chosen contact time, 1.0 ml of the test mixture was transferred to a tube containing 8.0 ml of neutralizer. After 5 minutes neutralization time, 1.0 ml of Nv-1 was post-inoculated into the neutralizer tube and incubated at 20° C. for 30 minutes. After incubation, 1.0 ml in duplicate was plated using either the pour or spread plate method.

Plates were allowed to set (or in the case of spread plates, dry) and incubated at the optimal growth temperature and duration until colonies were countable.

Yeast strains, Candida albicans ATCC #10231 and Candida tropicalis ATCC #750, were used.

Bacterial working cultures were inoculated from stock culture (Microbank bead) stored at −20° C. Subcultures were prepared by streaking for single colonies onto a QC controlled TSA (or required media) plate. After 18 to 24 hrs, a second subculture was prepared by streaking a single pure colony over the entire surface of a TSA (or required media) plate and incubating at 37±1° C. (or required optimal growth temperature) for 18 to 24 hrs. From this second subculture, a third subculture could if necessary, be produced by transferring a loopful of the culture to a third TSA (or required media) plate. The second and/or third subculture were the working culture(s).

To prepare the working culture of Candida sp., a subculture from the stock culture (MicroBank bead) was made by streaking for single colonies onto Malt extract agar (MEA) plates and incubating at 30±1° C. After 24 to 48 hrs a second subculture was prepared by streaking a single pure colony over the entire surface of an MEA plate and incubating for 24 to 48 hrs at 30±1° C. From this second subculture, a third subculture could, if necessary, be produced in the same way. The second and/or third subculture were the working culture(s).

Preparation of product test solutions: Test products are used at 100% V/V only and were tested in triplicate. No dilution of the test product was performed.

### Study Design:

The kill-time was set up to perform determinations at 15, 30 and 60 seconds in triplicate experiments for each of four formulations. The kill-time assays were set up to demonstrate up to a>6.0 log₁₀ kill. The final inoculum concentration was approximately 5.0×10⁷ cfu/ml. The test suspension (‘N’) was prepared to approximately 5.0×10⁹ cfu/ml to account for dilution in test. The test was performed in triplicate.

For counting of the test suspension 10′ and 10⁻⁸ dilutions of the test suspension were prepared and plated, 1.0 ml in duplicate of each.

Validation suspension (Nv) was used for the test controls. To make the microbial validation suspension (Nv), a ¼ dilution of the 10-5 dilution was made. To count, a 1:100 dilution was made (Nv⁻¹ and Nv⁻²) and 1.0 ml of Nv⁻² plated in duplicate.

### Study Procedure:

For the t=0 seconds time point for each replicate, 9.9 ml of Tryptone sodium chloride diluent was dispensed into a sterile tube. A 0.1 ml volume of N was added to the first replicate tube, mixed thoroughly, 0.1 ml was removed from the test tube and added to 0.9 ml of neutraliser. After 5 minutes neutralisation time, this sample (10-1) was then diluted to 10⁻⁵. A 0.1 ml volume from all dilutions (10⁻¹-10⁻⁵) were plated in duplicate using the appropriate media for the organism under test.

For each test replicate, a 9.9 ml of the sample under test was dispensed into a sterile tube. A 0.1 ml volume of N was added to the first replicate tube. This was mixed and a stopwatch immediately started. After the contact time, of t=15 seconds, t=30 seconds and t=60 seconds, 0.1 ml was removed from the test tube and added to 0.9 ml of neutralizer. After 5 minutes neutralization time, this sample (10⁻′) was then diluted to 10⁻⁵. A 0.1 ml volume from all dilutions (10⁻′-10⁻⁵) were plated in duplicate using the appropriate media for the organism under test. This procedure was repeated at timed intervals for each contact time and replicate.

### Results:

5.0% PVP-I antiseptic spray: The majority of strains tested against this formulation showed a>6 log₁₀ reduction in viability after 15 seconds. Several strains showed increased resistance to the formulation. Results include those presented below:
>6 log₁₀ (30 seconds): Escherichia coli; Salmonella enterica; Staphylococcus aureus, MRSA.; Staphylococcus epidermidis; Staphylococcus saprophyticus;
>6 log₁₀ (60 seconds): Enterococcus faecalis; Enterococcus faecium VRE; Micrococcus yunnanensis; Staphylococcus aureus; Staphylococcus epidermidis MRSE; Staphylococcus haemolyticus;
>5 log₁₀ (60 seconds): Staphylococcus aureus; Staphylococcus aureus; Enterococcus faecalis VRE, MDR.

A maximum kill from a suspension at approx. 7.0×108 colony forming units/ml recorded from 15-second contact.

The 5.0% PVP-I antiseptic spray, i.e., Betadine® spray, comprises poloxamer 407 (at 0.250% w/v) and glycerin.

10.0% PVP-I antiseptic solution: The majority of strains tested against this formulation showed a >6 log 10 reduction in viability after 15 seconds. Several strains showed higher resistance to this formulation. Sample results are presented as follows:
>6 log₁₀ (30 seconds): Enterococcus faecalis; Listeria monocytogenes; Staphyloccus aureus, MRSA; Staphylococcus epidermidis;
>6 log₁₀ (60 seconds): Enterococcus faecium VRE; Micrococcus yunnanensis;
>5 log₁₀ (60 seconds): Enterococcus faecalis; Staphylococcus epidermidis MRSE;

A maximum kill from a suspension at approx. 7.0×10⁸ colony forming units/ml recorded from 15-second contact.

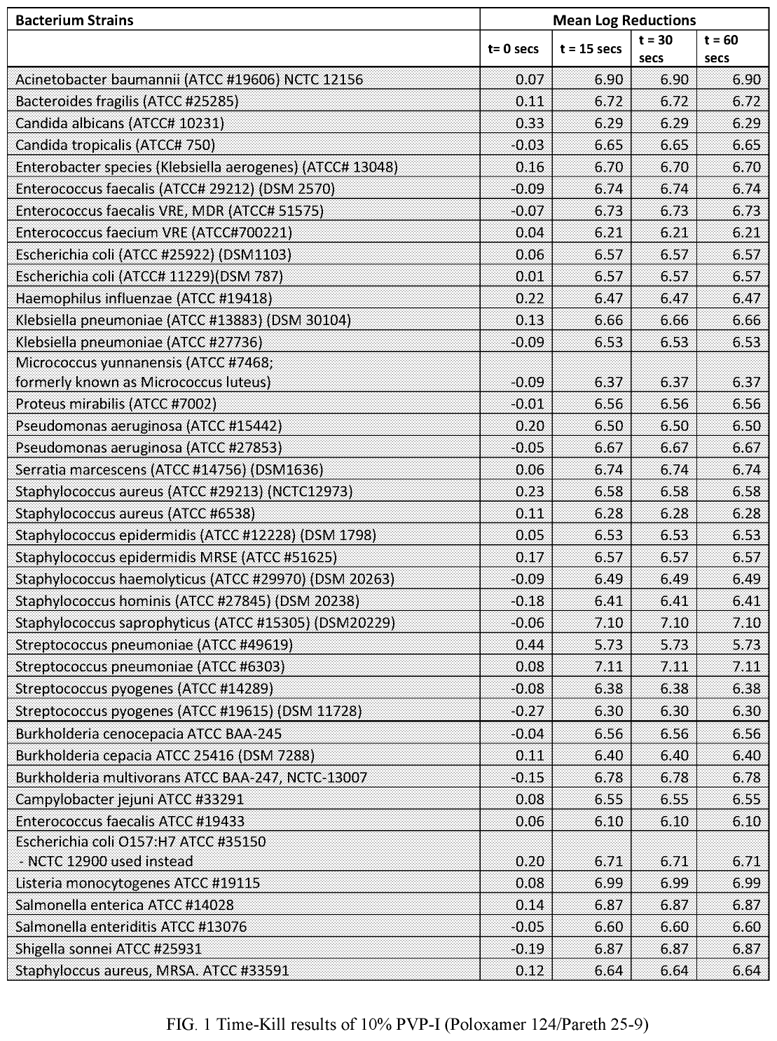

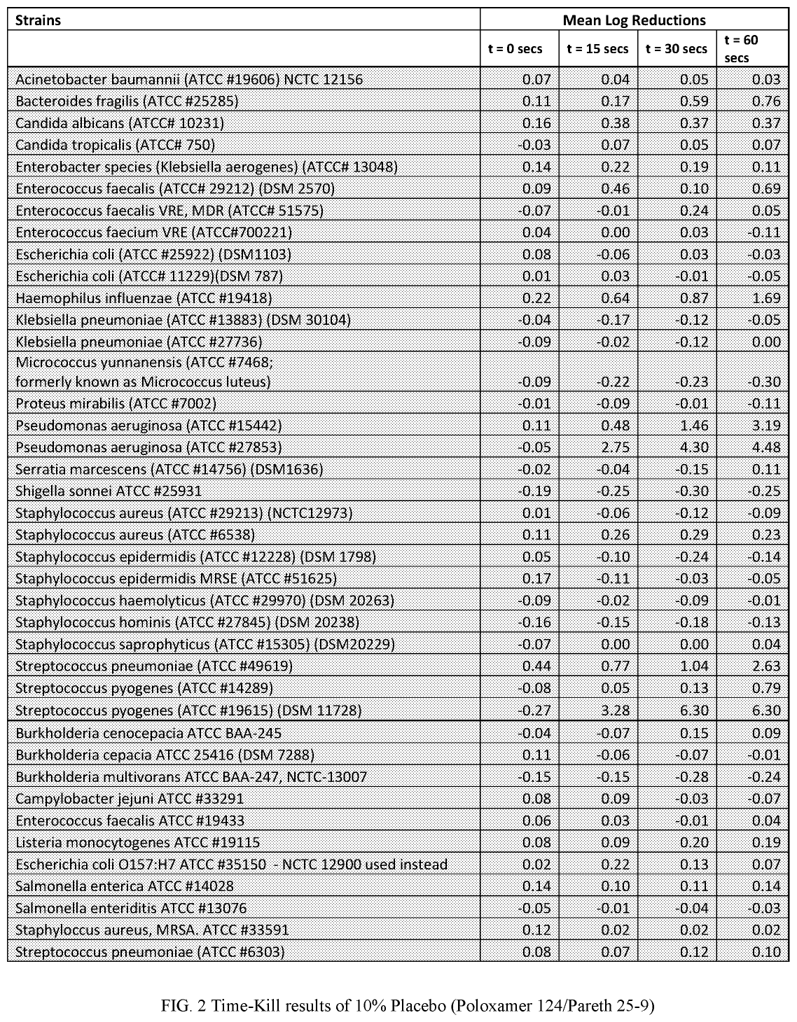

10% PVP-I (Poloxamer 124/Pareth 25-9) formulation: All of the strains tested against this formulation showed a>6 log₁₀ reduction in viability after 15 seconds (see FIG. 1). With Streptococcus pneumoniae, a maximum kill from a suspension at approx. 7.0×10⁸ colony forming units/ml was recorded from 15-second contact.
Placebo (Poloxamer 124/Pareth 25-9) formulation: This formulation was a no-active negative control. No antimicrobial efficacy was observed after 60 seconds, with the exception of three strains: Pseudomonas aeruginosa, Streptococcus pyogenes, Streptococcus pneumoniae (see FIG. 2). It is suspected that these particular strains may have been susceptible to co-formulants, e.g., detergent.

### Example 4. In-Vitro Time-Kill Evaluation Against Four Challenge Bacterial Species

This study used an In-vitro Time-Kill Method to evaluate the antimicrobial properties of several formulations when challenged with suspensions of Enterococcus faecalis (ATCC #29212), Enterococcus faecium (ATCC #700221), Staphylococcus aureus (ATCC #6538), and Staphylococcus epidermidis (ATCC #12228). This procedure was based upon the methodology described in ASTM E2783-11(2016), Standard Test Method for Assessment of Antimicrobial Activity for Water Miscible Compounds using a Time-Kill Procedure. All testing was performed in accordance with Good Laboratory Practices (as specified in 21 CFR Part 58). The percent and log₁₀ reduction in the microbial population of each challenge strain was determined following exposure to each test product at room temperature for 15, 30, and 60 seconds. All agar-plating was performed in duplicate.

It was noted that during the course of the Time-Kill evaluation, there was product inhibition noted at the 10¹ plated dilution for Staphylococcus aureus (ATCC #6538) and Staphylococcus epidermidis (ATCC #12228) versus 10% Professional Betadine® formulation and for Staphylococcus epidermidis (ATCC #12228) versus 10% Consumer PVP-I Betadine® formulation. This resulted in the 10⁻² plated dilution being the most reliable dilution for calculations for these test product/challenge microorganism combinations.

The Time-Kill evaluation results on 15 seconds post exposure against the 4 challenge bacterial species on some 10% PVP-I formulations of this disclosure as well as on a couple of commercial formulations are presented in Table 4 as follows:

TABLE 4
In-vitro Time-Kill Results at 15 seconds post exposure

| 10% PVP-I | | | Log reduction after 15 sec exposure | | | |
|---|---|---|---|---|---|---|
| formulations | | | Enterococcus | Enterococcus | Staphylococcus | Staphylococcus |
| (commercial | | | faecalis | faecium | aureus | epidermidis |
| source, or | | Viscosity | (ATCC | (ATCC | (ATCC | (ATCC |
| surfactants) | pH | (cps) | #29212) | #700221) | #6538) | #12228) |
| Betadine ® | 3.68 | 5.8 | .06 | 0.00 | 2.13 | 4.15 |
| Professional | | | | | | |
| Betadine® | 4.36 | 6.7 | .06 | .04 | 1.58 | 1.30 |
| Consumer | | | | | | |
| PVP-I | 4.21 | 7.0 | 6.76 | 5.98 | 6.22 | 6.43 |
| formulation | | | | | | |
| (Poloxamer 407 + | | | | | | |
| Pareth 25-9) | | | | | | |
| PVP-I | 4.06 | 6.3 | 6.76 | 5.98 | 6.22 | 6.43 |
| formulation | | | | | | |
| (Poloxamer 124 + | | | | | | |
| Pareth 25-9) | | | | | | |
| PVP-I | 4.16 | 7.2 | 6.76 | 5.98 | 6.22 | 6.43 |
| formulation | | | | | | |
| (SLS + Pareth | | | | | | |
| 25-9) | | | | | | |
| PVP-I | 4.16 | 7.3 | 1.55 | 1.47 | 6.22 | 6.43 |
| formulation | | | | | | |
| (Poloxamer 407 + | | | | | | |
| PEG 300) | | | | | | |
| (Poloxamer 407 + | 4.14 | 7.1 | 1.36 | 1.73 | 6.22 | 6.43 |
| propylene glycol) | | | | | | |

The 10% Betadine® professional formulation contains Pareth 25-9, purified water, and sodium hydroxide, in addition to povidone-iodine, while the 10% Betadine® consumer formulation contains glycerol and nonoxynol-9 as the surfactants. The results of these two formulations against the four challenge microorganisms 15 seconds, 30 seconds, and 60 seconds post exposure are presented in Tables 5 and 6, respectively:

TABLE 5
Time-Kill results for Betadine ® 10% PVP-I Professional Formulation

| | | | | Post | | |
|---|---|---|---|---|---|---|
| | Initial | Numbers | | Exposure | | |
| Challenge | Population | Control | Exposure | Population | log₁₀ | Percent |
| Microorganisms | (log₁₀) | (CFU/mL) | Time | (CFU/mL) | Reduction | Reduction |
| Enterococcus faecalis | 9.81 | 5.70 × 10⁷ | 15 seconds | >5.00 × 10⁷ | 0.06 | 12.28% |
| (ATCC #29212) | | | 30 seconds | >4.98 × 10⁷ | 0.06 | 12.63% |
| | | | 60 seconds | 2.88 × 10⁷ | 0.30 | 49.47% |
| Enterococcus faecium | 8.96 | 9.65 × 10⁶ | 15 seconds | 1.01 × 10⁷ | 0.00 | 0.00% |
| (ATCC #700221) | | | 30 seconds | 5.35 × 10⁶ | 0.25 | 44.56% |
| | | | 60 seconds | 1.38 × 10⁵ | 1.84 | 98.57% |
| Staphylococcus aureus | 9.21 | 1.65 × 10⁷ | 15 seconds | 1.24 × 10⁵ | 2.13 | 99.25% |
| (ATCC #6538) | | | 30 seconds | 7.80 × 10³ | 3.33 | 99.95% |
| | | | 60 seconds | <1.00 × 10² | 5.22 | 99.99% |
| Staphylococcus epidermidis | 9.40 | 2.72 × 10⁷ | 15 seconds | 1.90 × 10³ | 4.15 | 99.99% |
| (ATCC #12228) | | | 30 seconds | 3.50 × 10² | 4.39 | 99.99% |
| | | | 60 seconds | <1.00 × 10² | 5.43 | 99.99% |

As demonstrated in Table 5, Betadine® 10% Professional formulation reduced the microbial populations of Enterococcus faecalis (ATCC #29212), by less than 1.0 log₁₀ following the 60-second time exposure. The microbial populations of Enterococcus faecium (ATCC #700221) were reduced by 1.84 log₁₀ following the 60-second time exposure. The microbial populations of Staphylococcus aureus (ATCC #6538) were reduced by 2.13, 3.33, and 5.22 log₁₀ following the 15-, 30-, and 60-second exposure times, respectively. The microbial populations of Staphylococcus epidermidis (ATCC #12228) were reduced by greater than 4.0 log₁₀ following the 15-second time exposure and maintained these reductions for the 30- and 60-second time exposures.

TABLE 6
Time-Kill results for 10% Betadine ® Consumer formulation

| | | | | Post | | |
|---|---|---|---|---|---|---|
| | Initial | Numbers | | Exposure | | |
| Challenge | Population | Control | Exposure | Population | log₁₀ | Percent |
| Microorganisms | (log₁₀) | (CFU/mL) | Time | (CFU/mL) | Reduction | Reduction |
| Enterococcus faecalis | 9.81 | 5.70 × 10⁷ | 15 seconds | >5.00 × 10⁷ | 0.06 | 12.28% |
| (ATCC #29212) | | | 30 seconds | >5.00 × 10⁷ | 0.06 | 12.28% |
| | | | 60 seconds | 4.25 × 10⁷ | 0.13 | 25.44% |
| Enterococcus faecium | 8.96 | 9.65 × 10⁶ | 15 seconds | 8.80 × 10⁶ | 0.04 | 8.81% |
| (ATCC #700221) | | | 30 seconds | 7.20 × 10⁶ | 0.12 | 25.39% |
| | | | 60 seconds | 4.20 × 10⁶ | 0.36 | 56.48% |
| Staphylococcus aureus | 9.21 | 1.65 × 10⁷ | 15 seconds | 4.40 × 10⁵ | 1.58 | 97.33% |
| (ATCC #6538) | | | 30 seconds | 1.37 × 10⁵ | 2.08 | 99.17% |
| | | | 60 seconds | 1.00 × 10¹ | 6.22 | 99.99% |
| Staphylococcus epidermidis | 9.40 | 2.72 × 10⁷ | 15 seconds | 1.35 × 10⁶ | 1.30 | 95.04% |
| (ATCC #12228) | | | 30 seconds | 1.15 × 10⁵ | 2.37 | 99.58% |
| | | | 60 seconds | 1.30 × 10³ | 4.32 | 99.99% |

As shown by the data in Table 6, 10% Betadine® consumer formulation reduced the microbial populations of Enterococcus faecalis (ATCC #29212) and Enterococcus faecium (ATCC #700221) by less than 1.0 log₁₀ following the 60-second time exposure. The microbial populations of Staphylococcus aureus (ATCC #6538) were reduced by 1.58, 2.08, and 6.22 log₁₀ following the 15-, 30-, and 60-second exposure times, respectively. The microbial populations of Staphylococcus epidermidis (ATCC #12228) were reduced by 1.30, 2.37, and 4.32 log₁₀ following the 15-, 30-, and 60-second exposure times, respectively.

The results of 10% PVP-I formulation (containing Poloxamer 407 and Pareth 25-9) against the four challenge microorganisms 15 seconds, 30 seconds, and 60 seconds post exposure are presented in the following table:

TABLE 7
Time-Kill results for 10% PVP-I (Poloxamer 407 and Pareth 25-9) formulation

| | | | | Post | | |
|---|---|---|---|---|---|---|
| | Initial | Numbers | | Exposure | | |
| Challenge | Population | Control | Exposure | Population | log₁₀ | Percent |
| Microorganisms | (log₁₀) | (CFU/mL) | Time | (CFU/mL) | Reduction | Reduction |
| Enterococcus faecalis | 9.81 | 5.70 × 10⁷ | 15 seconds | <1.00 × 10¹ | 6.76 | 99.99% |
| (ATCC #29212) | | | 30 seconds | <1.00 × 10¹ | 6.76 | 99.99% |
| | | | 60 seconds | <1.00 × 10¹ | 6.76 | 99.99% |
| Enterococcus faecium | 8.96 | 9.65 × 10⁶ | 15 seconds | <1.00 × 10¹ | 5.98 | 99.99% |
| (ATCC #700221) | | | 30 seconds | <1.00 × 10¹ | 5.98 | 99.99% |
| | | | 60 seconds | <1.00 × 10¹ | 5.98 | 99.99% |
| Staphylococcus aureus | 9.21 | 1.65 × 10⁷ | 15 seconds | <1.00 × 10¹ | 6.22 | 99.99% |
| (ATCC #6538) | | | 30 seconds | <1.00 × 10¹ | 6.22 | 99.99% |
| | | | 60 seconds | <1.00 × 10¹ | 6.22 | 99.99% |
| Staphylococcus epidermidis | 9.40 | 2.72 × 10⁷ | 15 seconds | <1.00 × 10¹ | 6.43 | 99.99% |
| (ATCC #12228) | | | 30 seconds | <1.00 × 10¹ | 6.43 | 99.99% |
| | | | 60 seconds | <1.00 × 10¹ | 6.43 | 99.99% |

The results presented in Table 7 show that 10% PVP-I (Poloxamer 407 and Pareth 25-9) formulation reduced the microbial populations of all challenge species by 5.98 or greater log₁₀ following the 15-second time exposure and maintained these levels of reductions for the 30- and 60-second time exposures.

The Time-Kill results of a 10% PVP-I formulation (containing SLS and Pareth 25-9) against the four challenge microorganisms 15 seconds, 30 seconds, and 60 seconds post exposure are presented in Table 8.

TABLE 8
Time-Kill results for 10% PVP-I (SLS and Pareth 25-9) formulation

| | | | | Post | | |
|---|---|---|---|---|---|---|
| | Initial | Numbers | | Exposure | | |
| Challenge | Population | Control | Exposure | Population | log₁₀ | Percent |
| Microorganisms | (log₁₀) | (CFU/mL) | Time | (CFU/mL) | Reduction | Reduction |
| Enterococcus faecalis | 9.81 | 5.70 × 10⁷ | 15 seconds | <1.00 × 10¹ | 6.76 | 99.99% |
| (ATCC #29212) | | | 30 seconds | <1.00 × 10¹ | 6.76 | 99.99% |
| | | | 60 seconds | <1.00 × 10¹ | 6.76 | 99.99% |
| Enterococcus faecium | 8.96 | 9.65 × 10⁶ | 15 seconds | <1.00 × 10¹ | 5.98 | 99.99% |
| (ATCC #700221) | | | 30 seconds | <1.00 × 10¹ | 5.98 | 99.99% |
| | | | 60 seconds | <1.00 × 10¹ | 5.98 | 99.99% |
| Staphylococcus aureus | 9.21 | 1.65 × 10⁷ | 15 seconds | <1.00 × 10¹ | 6.22 | 99.99% |
| (ATCC #6538) | | | 30 seconds | <1.00 × 10¹ | 6.22 | 99.99% |
| | | | 60 seconds | <1.00 × 10¹ | 6.22 | 99.99% |
| Staphylococcus epidermidis | 9.40 | 2.72 × 10⁷ | 15 seconds | <1.00 × 10¹ | 6.43 | 99.99% |
| (ATCC #12228) | | | 30 seconds | <1.00 × 10¹ | 6.43 | 99.99% |
| | | | 60 seconds | <1.00 × 10¹ | 6.43 | 99.99% |

As shown in Table 8, the 10% PVP-I (SLS and Pareth 25-9) formulation reduced the microbial populations of all challenge species by greater than or equal to 5.98 log₁₀ following the 15-second time exposure and maintained these levels of reductions for the 30- and 60-second time exposures.

The Time-Kill results of a 10% PVP-I formulation (containing Poloxamer 407 and propylene glycol, as “PG”) against the four challenge microorganisms 15 seconds, 30 seconds, and 60 seconds post exposure are presented in Table 9.

TABLE 9
Time-Kill results for 10% PVP-I (Poloxamer 407 and PG) formulation

| | | | | Post | | |
|---|---|---|---|---|---|---|
| | Initial | Numbers | | Exposure | | |
| Challenge | Population | Control | Exposure | Population | log₁₀ | Percent |
| Microorganisms | (log₁₀) | (CFU/mL) | Time | (CFU/mL) | Reduction | Reduction |
| Enterococcus faecalis | 9.31 | 5.70 × 10⁷ | 15 seconds | 2.51 × 10⁶ | 1.36 | 95.60% |
| (ATCC #29212) | | | 30 seconds | 3.00 × 10² | 5.28 | 99.99% |
| | | | 60 seconds | <1.00 × 10¹ | 6.76 | 99.99% |
| Enterococcus faecium | 8.96 | 9.65 × 10⁶ | 15 seconds | 1.76 × 10³ | 1.73 | 98.18% |
| (ATCC #700221) | | | 30 seconds | 1.95 × 10² | 4.69 | 99.99% |
| | | | 60 seconds | <1.00 × 10¹ | 5.98 | 99.99% |
| Staphylococcus aureus | 9.21 | 1.65 × 10⁷ | 15 seconds | <1.00 × 10¹ | 6.22 | 99.99% |
| (ATCC #6538) | | | 30 seconds | <1.00 × 10¹ | 6.22 | 99.99% |
| | | | 60 seconds | <1.00 × 10¹ | 6.22 | 99.99% |
| Staphylococcus epidermidis | 9.40 | 2.72 × 10⁷ | 15 seconds | <1.00 × 10¹ | 6.43 | 99.99% |
| (ATCC #12228) | | | 30 seconds | <1.00 × 10¹ | 6.43 | 99.99% |
| | | | 60 seconds | <1.00 × 10¹ | 6.43 | 99.99% |

As shown in Table 9, the formulation (containing Poloxamer 407 and PG) reduced the microbial populations of Enterococcus faecalis (ATCC #29212) and Enterococcus faecium (ATCC #700221) by greater than 4.69 log₁₀ following the 30-second time exposure and maintained these levels of reductions for the 60-second time exposure. The microbial populations for Staphylococcus aureus (ATCC #6538) and Staphylococcus epidermidis (ATCC #12228) were reduced greater than or equal to 6.22 log₁₀ following the 15-second time exposure and maintained these levels of reductions for the 30- and 60-second time exposures.

The Time-Kill results of a 10% PVP-I (containing Poloxamer 124 and Pareth 25-9) formulation against the four challenge microorganisms 15 seconds, 30 seconds, and 60 seconds post exposure are presented in Table 10.

TABLE 10
Time-Kill results for 10% PVP-I (Poloxamer 124 and Pareth 25-9) formulation

| | | | | Post | | |
|---|---|---|---|---|---|---|
| | Initial | Numbers | | Exposure | | |
| Challenge | Population | Control | Exposure | Population | log₁₀ | Percent |
| Microorganisms | (log₁₀) | (CFU/mL) | Time | (CFU/mL) | Reduction | Reduction |
| Enterococcus faecalis | 9.81 | 5.70 × 10⁷ | 15 seconds | <1.00 × 10¹ | 6.76 | 99.99% |
| (ATCC #29212) | | | 30 seconds | <1.00 × 10¹ | 6.76 | 99.99% |
| | | | 60 seconds | <1.00 × 10¹ | 6.76 | 99.99% |
| Enterococcus faecium | 8.96 | 9.65 × 10⁶ | 15 seconds | <1.00 × 10¹ | 5.98 | 99.99% |
| (ATCC #700221) | | | 30 seconds | <1.00 × 10¹ | 5.98 | 99.99% |
| | | | 60 seconds | <1.00 × 10¹ | 5.98 | 99.99% |
| Staphylococcus aureus | 9.21 | 1.65 × 10⁷ | 15 seconds | <1.00 × 10¹ | 6.22 | 99,99% |
| (ATCC #6538) | | | 30 seconds | <1.00 × 10¹ | 6.22 | 99.99% |
| | | | 60 seconds | <1.00 × 10¹ | 6.22 | 99.99% |
| Staphylococcus epidermidis | 9.40 | 2.72 × 10⁷ | 15 seconds | <1.00 × 10¹ | 6.43 | 99.99% |
| (ATCC #12228) | | | 30 seconds | <1.00 × 10¹ | 6.43 | 99.99% |
| | | | 60 seconds | <1.00 × 10¹ | 6.43 | 99.99% |

The results in Table 10 show that the 10% PVP-I (Poloxamer 124 and Pareth 25-9) formulation reduced the microbial populations of all challenge species by greater than or equal to 5.98 log₁₀ following the 15-second time exposure and maintained these levels of reductions for the 30- and 60-second time exposures.

The Time-Kill results of a 10% PVP-I (containing Poloxamer 407 and PEG300) formulation against the four challenge microorganisms 15 seconds, 30 seconds, and 60 seconds post exposure are presented in Table 11.

TABLE 11
Time-Kill results for 10% PVP-I (Poloxamer 407 and PEG3 00) formulation

| | | | | Post | | |
|---|---|---|---|---|---|---|
| | Initial | Numbers | | Exposure | | |
| Challenge | Population | Control | Exposure | Population | log₁₀ | Percent |
| Microorganisms | (log₁₀) | (CFU/mL) | Time | (CFU/mL) | Reduction | Reduction |
| Enterococcus faecalis | 9.81 | 5.70 × 10⁷ | 15 seconds | 1.63 × 10⁶ | 1.55 | 97.14% |
| (ATCC #29212) | | | 30 seconds | <1.00 × 10¹ | 6.76 | 99.99% |
| | | | 60 seconds | <1.00 × 10¹ | 6.76 | 99.99% |
| Enterococcus faecium | 8.96 | 9.65 × 10⁶ | 15 seconds | 3.20 × 10⁵ | 1.47 | 96.68% |
| (ATCC #700221) | | | 30 seconds | 1.55 × 10² | 4.79 | 99.99% |
| | | | 60 seconds | <1.00 × 10¹ | 5.98 | 99.99% |
| Staphylococcus aureus | 9.21 | 1.65 × 10⁷ | 15 seconds | <1.00 × 10¹ | 6.22 | 99.99% |
| (ATCC #6538) | | | 30 seconds | <1.00 × 10¹ | 6.22 | 99.99% |
| | | | 60 seconds | <1.00 × 10¹ | 6.22 | 99.99% |
| Staphylococcus epidermidis | 9.40 | 2.72 × 10⁷ | 15 seconds | <1.00 × 10¹ | 6.43 | 99.99% |
| (ATCC #12228) | | | 30 seconds | <1.00 × 10¹ | 6.43 | 99.99% |
| | | | 60 seconds | <1.00 × 10¹ | 6.43 | 99.99% |

The 10% PVP-I (Poloxamer 407+PEG300) formulation reduced the microbial populations of Enterococcus faecalis (ATCC #29212) and Enterococcus faecium (ATCC #700221) by greater than 4.79 log₁₀ following the 30-second time exposure and maintained these levels of reductions for the 60-second time exposure. The microbial populations for Staphylococcus aureus (ATCC #6538) and Staphylococcus epidermidis (ATCC #12228) were reduced greater than or equal to 6.22 log₁₀ following the 15-second time exposure and maintained these levels of reductions for the 30- and 60-second time exposures.

### Example 5. Virucidal Assay Against SARS-COV-2 Viruses

### Virus, Media and Cells:

SARS-CoV-2, USA-WA1/2020 strain, virus stock was prepared prior to testing by growing in Vero 76 cells. Culture media for prepared stock (test media) was MEM with 2% fetal bovine serum (FBS) and 50 μg/mL gentamicin.

### Virucidal Assay:

Three test formulations (7.5% aqueous PVP-I formulation; 10% PVP-I/Poloxamer 124/Pareth 25-9 formulation; and Poloxamer 124/Pareth 25-9 Placebo formulation) in liquid form were mixed directly with virus solution so that the final concentration was 50% of each individual test formulation and 50% virus solution. A single concentration of each test formulation was tested in triplicate. Test media without virus was added to two tubes of the formulations to serve as toxicity and neutralization controls. Ethanol (70%) was tested in parallel as a positive control and water only as a virus control.

The test solutions and virus were incubated at room temperature (22±2° C.) for 15, 30, 60 seconds and 5 minutes. The solutions were then neutralized by a 1/10 dilution in MEM 10% FBS, 50 μg/mL gentamicin.

Surviving virus from each sample was quantified by standard end-point dilution assay. Briefly, the neutralized samples were pooled and serially diluted using eight log dilutions in test medium. Then 100 μL of each dilution was plated into quadruplicate wells of 96-well plates containing 80-90% confluent Vero 76 cells. The toxicity controls were added to an additional 4 wells of Vero 76 cells and 2 of those wells at each dilution were infected with virus to serve as neutralization controls, ensuring that residual sample in the titer assay plate did not inhibit growth and detection of surviving virus. Plates were incubated at 37±2° C. with 5% CO₂ for 5 days. Each well was then scored for presence or absence of infectious virus. The titers were measured using a standard endpoint dilution 50% cell culture infectious dose (CCID₅₀) assay calculated using the Reed-Muench (1948) equation and the log reduction value (LRV) of each compound compared to the negative (water) control was calculated.

### Results:

Virus titers and LRV of SARS-CoV-2 when incubated with a single concentration of each solution for the 4 indicated time points are shown in Table 12.

Note: “Pareth” in the table stands for Pareth 25-9.

TABLE 12
Virucidal efficacy of test solutions against SARS-CoV-2

| | Tested conc. | Incubation time | Virus titer^{a} | LRV^{b} |
|---|---|---|---|---|
| 7.5% aqueous PVP-I | 50% | 15 seconds | <0.67 | >4.0 |
| 10% PVP-I | 50% | 15 seconds | <0.67 | >4.0 |
| Poloxamer-124/Pareth | | | (TOX) | |
| Placebo | 50% | 15 seconds | 1.5 | 3.17 |
| Poloxamer-124/Pareth | | | (TOX) | |
| Ethanol | 50% | 15 seconds | <0.67 | >4.0 |
| Virus Control | na | 15 seconds | 4.67 | |
| 7.5% aqueous PVP-I | 50% | 30 seconds | <0.67 | >3.83 |
| 10% PVP-I | 50% | 30 seconds | <0.67 | >3.83 |
| Poloxamer-124/Pareth | | | (TOX) | |
| Placebo | 50% | 30 seconds | 2.0 | 2.5 |
| Poloxamer-124/Pareth | | | (TOX) | |
| Ethanol | 50% | 30 seconds | <0.67 | >3.83 |
| Virus Control | na | 30 seconds | 4.5 | |
| 7.5% aqueous PVP-I | 50% | 60 seconds | <0.67 | >4.0 |
| 10% PVP-I | 50% | 60 seconds | <0.67 | >4.0 |
| Poloxamer-124/Pareth | | | (TOX) | |
| Placebo | 50% | 60 seconds | <0.67 | >4.0 |
| Poloxamer-124/Pareth | | | (TOX) | |
| Ethanol | 50% | 60 seconds | <0.67 | >4.0 |
| Virus Control | na | 60 seconds | 4.67 | |
| 7.5% aqueous PVP-I | 50% | 5 minutes | <0.67 | >3.83 |
| 10% PVP-I | 50% | 5 minutes | <0.67 | >3.83 |
| Poloxamer-124/Pareth | | | (TOX) | |
| Placebo | 50% | 5 minutes | <0.67 | >3.83 |
| Poloxamer-124/Pareth | | | (TOX) | |
| Ethanol | 50% | 5 minutes | <0.67 | >3.83 |
| Virus Control | na | 5 minutes | 4.5 | |

^{a}Log₁₀ CCID₅₀ of virus per 0.1 mL. The lower limit of detection is 0.67 Log₁₀ CCID₅₀/0.1 mL
^{b}LRV (log reduction value) is the reduction of virus compared to the virus control

In vitro virus tests are reliant on host cells for virus growth, that is, viruses can only grow in a host cell, which means that host cells are needed for studies evaluating viruses. In the studies presented above, it has been observed that some formulations, the ones with surfactants, have demonstrated cell toxicity (marked with “TOX” in the table), that is, these formulations killed the host cells that contained the virus and that otherwise allowed the virus to grow.

While the subject matter of this disclosure has been described with reference to illustrative embodiments and examples, the description is not intended to be construed in a limiting sense. Thus, various modifications of the illustrative embodiments, as well as other embodiments of the invention, will be apparent to persons skilled in the art upon reference to this description. It is therefore contemplated that the appended claims will cover any such modifications or embodiments.

All publications, patents and patent applications referred to herein are incorporated by reference in their entirety to the same extent as if each individual publication, patent or patent application was specifically and individually indicated to be incorporated by reference in its entirety.

1. (canceled)

2. The aqueous pharmaceutical preparation of claim 28, wherein said aqueous pharmaceutical preparation is a topical preparation.

3. The aqueous pharmaceutical preparation of claim 28, wherein said aqueous pharmaceutical preparation is a scrub or a spray.

4. (canceled)

5. The aqueous pharmaceutical preparation of claim 33, wherein said alcohol ethoxylate is Pareth 25-9.

6. (canceled)

7. The aqueous pharmaceutical preparation of claim 33, wherein the poloxamer is selected from the group of poloxamer 124, poloxamer 182, poloxamer 188, poloxamer 331, and poloxamer 407, or a combination thereof.

8. The aqueous pharmaceutical preparation of any claim 7, wherein the poloxamer comprises poloxamer 124 or poloxamer 407, or a combination thereof.

9. The aqueous pharmaceutical preparation of claim 8, wherein the poloxamer comprises poloxamer 124.

10. The aqueous pharmaceutical preparation of claim 28, wherein said pharmaceutical preparation comprises SLS.

11. The aqueous pharmaceutical preparation of claim 28, wherein said pharmaceutical preparation comprises polyoxypropylene, and said polyoxypropylene has a molecular mass at about 1200 to about 4000 g/mol.

12. The aqueous pharmaceutical preparation of claim 11, wherein said polyoxypropylene comprises a content of polyoxyethylene ranging from about 40 to about 70%.

13. The aqueous pharmaceutical preparation of claim 28, wherein the aqueous pharmaceutical preparation comprises about 0.2% w/v of the alcohol ethoxylate.

14. The aqueous pharmaceutical preparation of claim 28, wherein the aqueous pharmaceutical preparation comprises about 0.1% w/v to about 0.5% w/v of the one or more surfactants.

15. The aqueous pharmaceutical preparation of claim 15, wherein the aqueous pharmaceutical preparation comprises about 0.2% w/v of the one or more surfactants.

16. The aqueous pharmaceutical preparation of claim 28, wherein said aqueous pharmaceutical preparation achieves a log reduction in a value of 6 or higher after 15 seconds exposure against two or more of said gram-positive bacteria in the time-kill study.

17. The aqueous pharmaceutical preparation of claim 16, wherein said aqueous pharmaceutical preparation achieves a log reduction in a value of 6 or higher after 15 seconds exposure against all of said gram-positive bacteria in the time-kill study.

18. The aqueous pharmaceutical preparation of claim 28, wherein said aqueous pharmaceutical preparation further demonstrates efficacy against SARS-CoV-2 viruses, wherein said efficacy is proven by a log reduction in a value of about 4 or higher after 15 seconds exposure in a time-kill study.

19. The aqueous pharmaceutical preparation of claim 28, wherein said aqueous pharmaceutical preparation comprises about 10% w/v povidone-iodine (PVP-I), about 0.2% w/v of Pareth 25-9, and about 0.2% w/v of poloxamer 124.

20. A method of reducing risk, incidence or severity of COVID-19 disease caused by SARS-CoV-2 viruses in a human subject, the method comprising topical application to the human subject an effective amount of the aqueous pharmaceutical preparation of claim 28, wherein the aqueous pharmaceutical preparation is applied prior to, during or after the human subject is exposed to SARS-CoV2 viruses or a person infected with SARS-CoV-2 viruses.

21-22. (canceled)

23. The method of claim 20, wherein the aqueous pharmaceutical preparation is topically applied to the skin of the human subject.

24-27. (canceled)

28. An aqueous pharmaceutical preparation comprising povidone-iodine (PVP-I) at a concentration of from about 0.5% to about 10% w/v and an effective amount of a combination of
1. an alcohol ethoxylate; and
2. one or more surfactants selected from the group of polyoxypropylene, polyoxyethylene, sodium lauryl sulfate (SLS), and poloxamer, or a combination thereof, and optionally
3. one or more viscosity modifying agents;
wherein the viscosity of said aqueous pharmaceutical preparation is within the range of about 2 cps to about 500 cps at 25° C., and
wherein said aqueous pharmaceutical preparation has efficacy against one or more gram-positive bacteria selected from the group of Enterococcus faecalis, Enterococcus faecium, Staphylococcus aureus, and Staphylococcus epidermidis, wherein said efficacy is demonstrated by a log reduction in a value of about 4 or higher after 15 seconds exposure in a standard time-kill study.

29. The aqueous pharmaceutical preparation of claim 28, wherein the aqueous pharmaceutical preparation is a topical preparation and comprises one or more viscosity modifying agents.

30. (canceled)

31. The aqueous pharmaceutical preparation of claim 29 or 30, wherein said one or more viscosity modifying agents comprises humectants selected from the group of celluloses, glycols, alpha hydroxy acids, polymeric polyols, lithium chloride, glyceryl triacetate, sugar alcohols, sodium hexametaphosphate, and a combination thereof.

32. The aqueous pharmaceutical preparation of claim 29, wherein said one or more viscosity modifying agents are selected from the group of hydroxyethyl cellulose, hydroxypropyl methyl cellulose, and a combination thereof.

33. The aqueous pharmaceutical preparation of claim 28, wherein the aqueous pharmaceutical preparation comprises a combination of an alcohol ethoxylate and a poloxamer.

34. The aqueous pharmaceutical preparation of claim 28, wherein the aqueous pharmaceutical preparation comprises about 10% w/v of PVP-I and one or more viscosity modifying agents, and wherein the viscosity of said aqueous pharmaceutical preparation is within the range of about 10 cps to about 300 cps at 25° C.

35. The aqueous pharmaceutical preparation of claim 34, wherein the one or more viscosity modifying agents comprise hydroxyethyl cellulose, and wherein the viscosity of said aqueous pharmaceutical preparation is about 20 cps at 25° C.

36. The aqueous pharmaceutical preparation of claim 34, wherein the one or more viscosity modifying agents comprise hydroxypropyl methyl cellulose, and wherein the viscosity of said aqueous pharmaceutical preparation is about 250 cps at 25° C.

37. A method of reducing an infection risk to a patient or a healthcare professional in a clinical setting, comprising topically applying an antiseptically effective amount of an aqueous pharmaceutical preparation of claim 28 to the skin of the patient or the healthcare professional, wherein the infection risk is posed by microorganisms or viruses.
